(19) United States
(12) Patent Application Publication
(10) Pub. No.: US 2012/0088976 A1
(43) Pub. Date: Apr. 12, 2012

(54) SYSTEM AND METHOD FOR SUCTION-ASSISTED OBJECT REMOVAL

(76) Inventor: Hassan Shehadeh, Freehold, NJ (US)

(21) Appl. No.: 13/331,009

(22) Filed: Dec. 20, 2011

Related U.S. Application Data
(63) Continuation-in-part of application No. 29/316,561, filed on Dec. 30, 2009.

(51) Int. Cl. A61M 1/00; A61B 1/227

(52) U.S. Cl. 600/187

(57) ABSTRACT

System and method to remove a foreign object from a body cavity, the system including: a speculum that has a base configured to attach to an otoscope head; a hollow tapered section, having a wide end coupled to the base, and a narrow end that is open; and a hollow tube having a proximal end and an open distal end; the proximal end coupled to the narrow end of the hollow tapered section to form a hollow interior of the speculum. The system further includes a suction port coupled to a lateral side of the speculum, the suction port configured to provide access from outside the speculum to the hollow interior of the speculum; a suction pump configured to provide a variably controlled suction strength; and an interface to provide the variably controlled level of suction to the suction port.

### CROSS-REFERENCE TO RELATED APPLICATIONS

This application is a Continuation-in-Part of U.S. Design Pat. No. application Ser. No. 29/316,561, filed on Dec. 30, 2009, the content of which is hereby incorporated by reference in its entirety.

### BACKGROUND

1. Field of the Invention

Embodiments of the present invention generally relate to suction-assisted removal of objects from body cavities. More specifically, embodiments of the present invention relate to a system and method for suction-assisted removal of a foreign object from an ear canal or from a nasal cavity.

2. Description of the Related Art

Body cavities such as the ear canal or nasal cavity at times may become at least partially obstructed, either by natural substances (e.g., earwax or mucus), by a foreign object that enters the body cavity, or by swelling of tissue. For example, a foreign object may enter the ear canal if a child places object in their ear or nose. Foreign objects may also enter the ear canal or nasal passage if a person suffers an accident or injury.

An otoscope or auriscope is a hand-manipulated medical device which is used to inspect body cavities such as the ear canal or nasal cavity. Health care providers may use an otoscope to examine the outer ear and middle ear for the presence of an obstruction. An otoscope includes a handle and a head. The head typically contains a light source and a simple low-power magnifying lens. The distal (front) end of the otoscope has an attachment for disposable plastic ear specula. The examiner first straightens the ear canal by pulling on the pinna and then inserts the ear speculum side of the otoscope into the external ear. The examiner can then look through a lens on the rear of the instrument and see inside the ear canal. In many models, the lens can be removed, which allows the examiner to insert instruments through the otoscope into the ear canal, such as for removing earwax (cerumen).

A pneumatic otoscope is similar to a regular otoscope, but it allows the health professional to give a gentle puff of air into the ear canal to see how the eardrum responds to a positive change in air pressure. The pneumatic otoscope has a pneumatic tube attached to the otoscope. The doctor squeezes a rubber bulb connected to the tube to positively change the pressure in the ear canal, testing how well the eardrum moves.

An otoscope may also be used to examine a patient's nose, avoiding the need for a separate nasal speculum, or for examination of the upper throat of a patient.

Apparatus are known in the art to remove obstructions and/or foreign objects from body cavities. Some such apparatus may employ a grasping technique, using a hook-like portion to grasp the foreign object, or otherwise reach around and pull out an obstruction. For example, the known art may include instruments that may have a plurality of flexible members that expand and attempt to grasp a foreign object. The known art may also include an ear speculum that incorporates one or more protrusions that move independently of the body of the speculum. The one or more protrusions may be curled or otherwise manipulated to grasp a foreign object.

The grasping technique of the known art suffers from a disadvantage by attempting to capture the object before removing it. The grasping technique, by using a hook or similar part that is positioned to go behind the foreign object in order to grasp it and pull it back, requires some clearance around at least a portion of the object in order for the hook to be able to go around the foreign object. However, often there is not adequate clearance around the foreign object to accommodate such a hook. The grasping technique often creates more problems than solutions because, while attempting to hook the object, the doctor or nurse risks pushing the object further down the ear canal. As the foreign object is pushed further down the ear canal, foreign object becomes more difficult to remove. As the object gets deeper into the canal, a more invasive medical procedure may be required.

Another method of the known art is to use a clip-like instrument, in which a tip of the clip-like instrument grabs the foreign object like a pliers, and then the foreign object is pulled out. This method may be useful for small objects for which the width of the ear canal does not present a significant constraint. However, if the object that is stuck in the ear canal is large enough, then the pliers might not be able to open wide enough inside the ear canal and there would be inadequate space on the side of the foreign object in order to grab it. Using the clip-like instrument presents the risk of pushing the object further down the ear canal and thus requiring a more invasive medical procedure.

Other apparatus known in the art to remove obstructions, foreign objects, and/or other material from body cavities may employ a suction technique. The suction technique may be used for stomach pumps, for devices designed to remove food lodged in a throat or airway, for devices to remove fluid from the lungs of a potential drowning victim, or abortion devices. Such devices typically require a high suction strength vacuum source and/or be able to remove a relatively large volume of material through a hollow tube. These devices are often designed to work within parts of the body that may experience involuntary contractions, such as the throat, trachea, stomach, lungs, etc., making it more difficult to remove a foreign object if contracting muscles either grasp the foreign object or make it more difficult to insert and operate a removal tool. Some devices, such as abortion devices, are designed to cut into tissue. For these reasons, a relatively high power suction is often required, as well as a relatively large suction tube that is able to withstand muscular pressures and is able to accommodate a relatively large size or quantity of material to be removed through the tube.

What is needed is an improved removal apparatus that can operate in a way that reduces the risk of pushing an obstruction and/or foreign object further into a body cavity, and with reduced risk of damage to sensitive and/or delicate body tissue.

### SUMMARY

Embodiments of the present invention relate to a system and method for suction-assisted removal of obstructions and/or foreign objects from body cavities such as the ear canal or nasal cavity, in a way that reduces the risk of pushing the obstructions and/or foreign objects further into the body cavity, and reduces the risk of damage to sensitive and/or delicate body tissue.

Obstructions and/or foreign objects may be referred to collectively herein as “foreign objects” unless the context clearly indicates otherwise.

Embodiments in accordance with the present invention provide a system to remove a foreign object from a body cavity, the system including: a speculum that has a base configured to attach to an otoscope head; a hollow tapered section, having a wide end coupled to the base, and a narrow end that is open; and a hollow tube having a proximal end and an open distal end; the proximal end coupled to the narrow end of the hollow tapered section to form a hollow interior of the speculum. The system further includes a suction port coupled to a lateral side of the speculum, the suction port configured to provide access from outside the speculum to the hollow interior of the speculum; a suction pump configured to provide a variably controlled suction strength; and an interface to provide the variably controlled level of suction to the suction port.

Embodiments in accordance with the present invention further provide a method to remove a foreign object from a body cavity, the method including the steps of: selecting a hollow tube having an inner diameter less than a size of the foreign object, the hollow tube having a proximal end and a distal end opposite from the proximal end; positioning the distal end of the hollow tube next to the foreign object; providing a variably controlled suction strength through the proximal end of hollow tube, wherein the suction strength is sufficient to hold the foreign object to the distal end of the hollow tube; and removing together the hollow tube and the foreign object at the distal end of the hollow tube, while maintaining the variably controlled suction strength.

### BRIEF DESCRIPTION OF THE DRAWINGS

So the manner in which the above recited features of the present invention can be understood in detail, a more particular description of embodiments of the present invention, briefly summarized above, may be had by reference to embodiments, which are illustrated in the appended drawings. It is to be noted, however, the appended drawings illustrate only typical embodiments encompassed within the scope of the present invention, and, therefore, are not to be considered limiting, for the present invention may admit to other equally effective embodiments, wherein:

FIG. 1 illustrates an otoscope as known in the art;

FIG. 2 illustrates a schematic of a system to remove a foreign object from a body cavity, in accordance with an embodiment of the present invention;

FIG. 3 illustrates a perspective view of a speculum in accordance with an embodiment of the present invention;

FIG. 4 illustrates a side plan view of a speculum in accordance with an embodiment of the present invention;

FIG. 5 illustrates a side sectional view of a speculum in accordance with an embodiment of the present invention;

FIG. 6 illustrates a detailed sectional view of an interface between a speculum and an otoscope head, in accordance with an embodiment of the present invention;

FIG. 7 illustrates a detailed top plan view of a speculum in accordance with an embodiment of the present invention;

FIG. 8 illustrates a less detailed top plan view of a speculum in accordance with an embodiment of the present invention;

FIG. 9 illustrates a detailed top plan view of a speculum in accordance with another embodiment of the present invention;

FIG. 10 illustrates a detailed top plan view of a speculum in accordance with another embodiment of the present invention;

The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include”, “including”, and “includes” mean including but not limited to. To facilitate understanding, like reference numerals have been used, where possible, to designate like elements common to the figures. Optional portions of the figures may be illustrated using dashed or dotted lines.

### DETAILED DESCRIPTION

Embodiments of the present invention generally relate to suction-assisted removal of objects from body cavities. More specifically, embodiments of the present invention relate to a system and method for suction-assisted removal of a foreign object from an ear canal or from a nasal cavity, such that the foreign object is held by suction force to the tip of a removal tool. The foreign object and the removal tool are then removed together from the body cavity while maintaining suction.

As used herein, the term “module” refers generally to a logical sequence or association of steps, processes or components. For example, a software module may comprise a set of associated routines or subroutines within a computer program. Alternatively, a module may comprise a substantially self-contained hardware device. A module may also comprise a logical set of processes irrespective of any software or hardware implementation.

An ear canal generally has a diameter of about 5 mm to about 10 mm, and a length from the eardrum to the pinna of about 31 mm to about 35 mm. A nasal cavity is relatively larger than the ear canal. The distance from the outer end of the nostril to the opening into the nasal cavity is approximately 40 mm to about 50 mm, with a diameter of an opening being about 2 mm to about 3 mm.

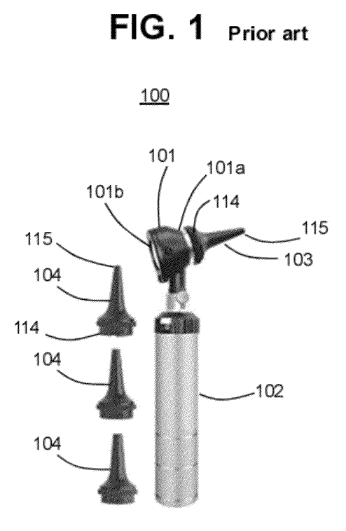

Embodiments in accordance with the present invention are usable together with an otoscope. FIG. 1 illustrates an otoscope 100 as known in the art. Otoscope 100 includes head 101 adjustably coupled to handle 102. Head 101 includes a forward side 101a to which a removable speculum 103 is detachably coupled to head 101. Head 101 also includes a rear side 101b opposite from forward side 101a. A plurality of specula 104 are shown detached from head 101. The coupling of speculum 103 or 104 to head 101 is known to persons of skill in the art, and includes one or more of a snap fit, a threaded screw fit, and a friction fit.

Speculum 103 and 104 includes a coupling section 114 that couples to head 101, and a tapered tip section 115 opposite from coupling section. Speculum 103 and 104 are open at both coupling section 114 and at the tip of tapered tip section 115, and speculum 103 and 104 are substantially hollow.

In operation of otoscope 100, speculum 103 is attached to head 101, and tapered tip 115 is inserted into an ear canal. A view of the ear canal as seen at tip 115 is presented through speculum 103 to a user observing from the rear side 101b of head 101. The image path may include lenses, or may include an image sensor on forward side 101a and an image display on rear side 101b.

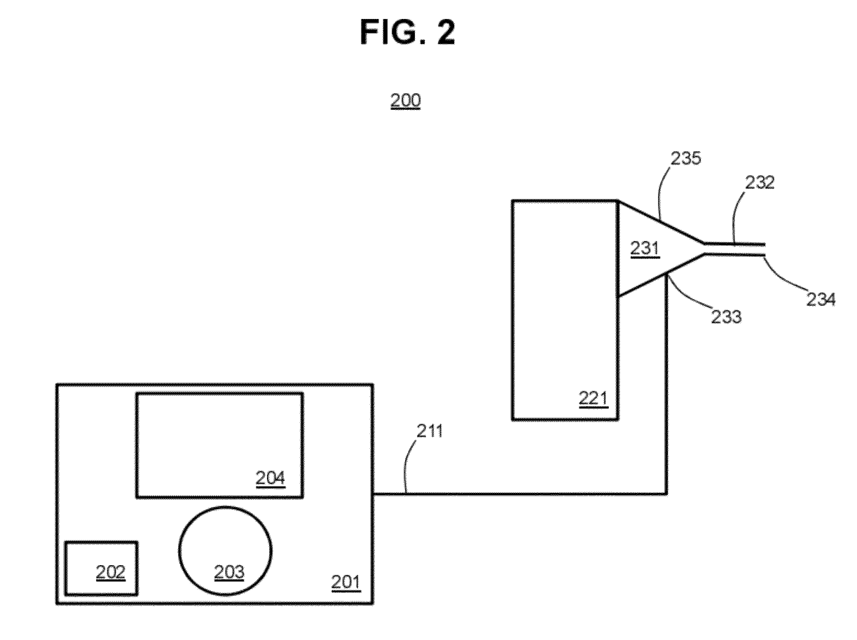

FIG. 2 is a schematic of system 200 in accordance with an embodiment of the present invention. System 200 includes a conventional otoscope 221 coupled to a speculum 231 that is designed in accordance with an embodiment of the present invention. Speculum 231 includes a conical portion 235 having a wide end and a narrow end, a hollow cylindrical portion 232 having a proximal end and a distal end 234, and a suction port 233. Hollow cylindrical portion 232 may be flexible in order to better accommodate turns in the ear canal or the nasal cavity. The narrow end of conical portion 235 is coupled to the proximal end of hollow cylindrical portion 232. The distal end 234 of hollow cylindrical portion 232 has an opening to the interior of hollow cylindrical portion 232. The interior of hollow cylindrical portion 232 is open from distal end 234 to suction port 233. Speculum 231 may be provided in a plurality of selectable sizes, the selectable sizes differing at least in the inner diameter of the hollow cylindrical portion 232. The selectable sizes may also differ in an outer diameter and/or a length, or combinations thereof. The outer diameter of hollow cylindrical portion 232 is selected to be approximately 4 mm, which is less than the diameter of a typical ear canal, thereby allowing for a flow of air from outside the ear canal, through a gap formed between the outer diameter of hollow cylindrical portion 232 and the inner wall of the ear canal, and to distal end 234.

Suction port 233 is coupled to a first end of suction tube 211. A second end of suction tube 211 is coupled to a variable suction pump 201. Variable suction pump 201 includes a power switch 202, a variable suction control 203, and a suction monitor 204. Variable suction control 203 allows for an amount or strength of suction to be variably controlled. Variable suction control 203 may be, for instance, a slide control, rotary control, an up/down digital control, and so forth. The amount or strength of the suction is indicated by suction monitor 204. Suction monitor 204 may be, for instance, a meter, an LED bar indicator, a digital display, and so forth. Suction tube 211 may be flexible, but having sufficient radial rigidity to substantially prevent collapse of suction tube 211 when subjected to the amount or strength of suction as described herein.

System 200 is designed to use suction to make the foreign object adhere to distal end 234 of hollow cylindrical portion 232, without being sucked through or entirely into portion 232. The foreign object is then removed when system 200 is removed from the body cavity while maintaining suction.

Operation of system 200 begins with a conventional speculum attached to otoscope 221, rather than speculum 231. Otoscope 221 with the conventional speculum is used as a guide by a medical attendant (e.g., nurse, doctor, etc.) to examine a foreign object inside the body cavity (e.g., ear canal or nasal cavity), for instance examining a size or a position of the foreign object. Once the foreign object is examined, otoscope 221 with conventional speculum is removed from the body cavity.

The conventional speculum is then replaced with speculum 231. The size of speculum 231 is selected based upon the size of the foreign object of the foreign object. In accordance with an embodiment of the present invention, a size of speculum 231 is selected in order to have the inner diameter of hollow cylindrical portion 232 to be approximately 60% to approximately 90% of the cross-sectional size of the foreign object. The inner diameter of hollow cylindrical portion 232 is selected to be small enough so that the foreign object will be coupled by suction to the distal end of hollow cylindrical portion 232 without being sucked through or entirely into hollow cylindrical portion 232. The inner diameter is also selected small enough so that the suction coupling of the foreign object to distal end 234 is not substantially negatively affected by a flow of air around the foreign object and into distal end 234. Conversely, the inner diameter is selected large enough so that sufficient suction force is exerted on the foreign object in order to dislodge and pull it out of the body cavity. In accordance with an embodiment of the present invention, system 200 may be designed to remove a foreign object having a cross-sectional size of at least 1 mm to about 5 mm. The resulting inner diameter of hollow cylindrical portion 232 may range from about 1.0 mm to about 3.0 mm.

Suction tube 211 is coupled to suction port 233 if suction tube 211 is not already so coupled. Distal end 234 is positioned up against the foreign object. Variable suction pump 201 is turned on. The strength of the suction may initially be set to a relatively low value in order to reduce risk to surrounding tissue. The strength of the suction is then increased to a sufficient level to dislodge and pull out the foreign object from the body cavity. More than one attempt may be necessary, at increasing suction levels, if initially the suction level is not adequate to dislodge and pull out the foreign object.

Embodiments in accordance with the present invention are used in proximity to delicate human tissue, therefore the amount of suction should be controlled and/or limited in order to prevent or minimize damage to surrounding human tissue. In accordance with an embodiment of the present invention, the suction level of system 200 may be controlled between a pressure of 0 mm of mercury (chemical symbol Hg) to about 1000 mm of Hg. The upper pressure limit of 1000 mg of Hg is approximately equal to the pressure exerted on an eardrum by a diver under about 10 meters of water.

Unlike pneumatic otoscopes, embodiments in accordance with the present invention suck air in through distal end 234 rather than discharge air out from distal end 234. Furthermore, pneumatic otoscopes provide a relatively brief puff of air at a low pressure in order to move the eardrum, often by use of a manually-operated bulb. In contrast, embodiments in accordance with the present invention provide an adjustably constant and relatively longer suction that is strong enough to dislodge and/or grip by use of suction a foreign object, well enough to hold the foreign object securely to distal end 234 as both distal end 234 and the foreign object are removed from the body cavity. The suction is provided by a powered pump, thereby being able to provide a more controlled suction strength over a longer period of time.

Persons of skill in the art will recognize that certain steps described above may be performed in a different order than described above, without departing from the spirit of the invention.

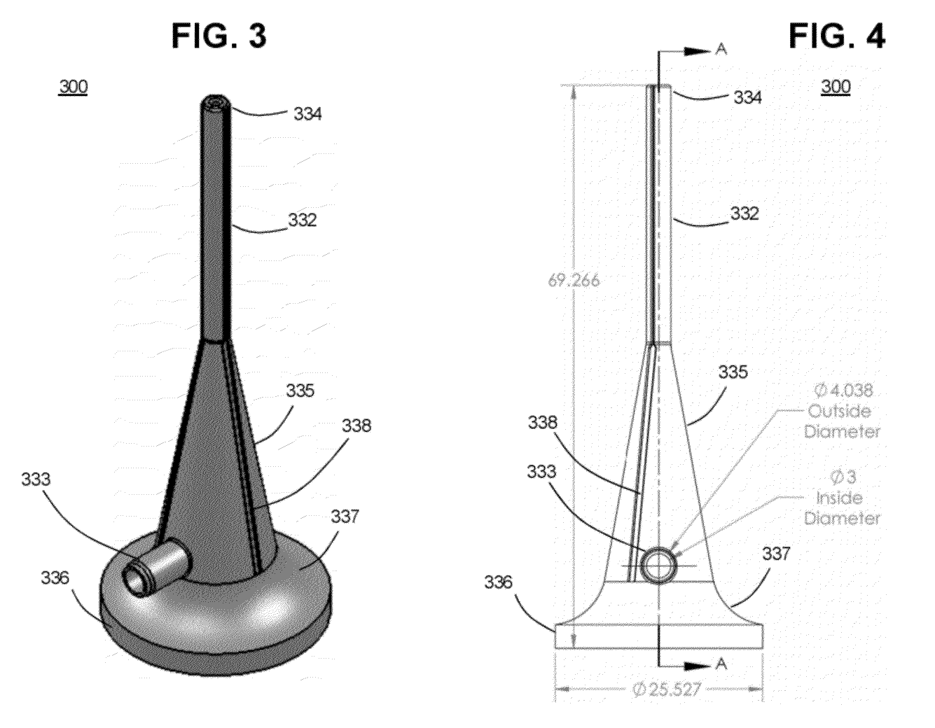

FIG. 3 illustrates a perspective view of a speculum 300 in accordance with an embodiment of the present invention. Structure and function of speculum 300 is similar to the structure and function of speculum 231 described above in connection with FIG. 2.

Speculum 300 includes a cylindrical base portion 336 having an open first end that is adapted to be coupled to an otoscope. A second end of cylindrical base portion 336 is arranged opposite from the open first end. Cylindrical base portion 336 has a substantially constant diameter and circumference from the open first end to the second end. The outer diameter of cylindrical base portion 336 is sized to fit different sizes of heads for commercially available otoscopes 100, and can range from about 25 mm to about 40 mm. The height of cylindrical base portion 336 may range from about 2 mm to about 3 mm.

Second end of cylindrical base portion 336 couples to a second tapered section 337. In the illustrated embodiment, second tapered section 337 is illustrated as a concavely-tapered section, but other shapes may be used, such as a conical taper or a convex taper or a more complex shape or an irregular shape. Second tapered section 337 has a first end having a circumference sized to match the circumference of cylindrical base portion 336, and a second end which has a relatively smaller diameter and circumference than the first end. Second tapered section 337 is hollow and open from the first end to the second end.

Second end of second tapered section 337 couples to a first end of first tapered section 335. First tapered section 335 has a first end having a circumference sized to match the circumference of the second end of second tapered section 337, and a second end which has a relatively smaller diameter and circumference than the first end. First tapered section 335 is proportionally less tapered than second tapered section 337. First tapered section 335 has a length of about 20 mm to about 30 mm. First tapered section 335 is hollow and open from the first end to the second end. First tapered section 335 further includes a suction port 333 that provides access through a sidewall of first tapered section 335 to the interior of first tapered section 335. Reducing the interior open volume of speculum 300 helps reduce the volume of air that is removed in order to maintain a desired suction strength.

Second end of first tapered section 335 couples to a hollow cylindrical portion 332 having a proximal end and a distal end 334. The distal end 334 of hollow cylindrical portion 332 has an opening to the interior of hollow cylindrical portion 332. The interior of hollow cylindrical portion 332 is open from distal end 334 to suction port 333. Cylindrical portion 332 may be available in a range of selectable lengths, from about 30 mm to about 100 mm. The length of cylindrical portion 332 may be selected on the basis of the depth of the body cavity where the foreign object is lodged, and which may be approximated by the age of the patient.

A plurality of grooves 338 or slots may be provided, running lengthwise on an outer surface of first tapered section 335 and hollow cylindrical portion 332. Grooves 338 function to help prevent an undue pressure loss or partial vacuum from forming in the ear canal, outside speculum 300, when system 100 is operated as described herein. Grooves 338 form a passageway for air to enter the ear canal and help equalize pressure when system 200 is operated.

Speculum 300 may be provided in a variety of sizes. For example, for usage in an ear canal, the length of hollow cylindrical portion 332 may be about 30 to about 40 mm, with an inner diameter of about 1.0 mm to about 3.0 mm. However, for usage in a nasal cavity, the length of hollow cylindrical portion 332 may be about 40 mm to about 50 mm, and the inner diameter may be about 2.0-3.0 mm.

Cylindrical base portion 336, second tapered section 337, first tapered section 335 and hollow cylindrical portion 332 each have a common major axis, and which is substantially the same as axis A-A illustrated in FIG. 4.

FIG. 4 illustrates an upright, side plan view of speculum 300 mated to otoscope head 501 with typical design dimensions, in accordance with an embodiment of the present invention. Persons of skill in the art will realize that even though design dimensions may be shown in FIG. 4 and other drawings herein as a single value, as-manufactured dimensions may have a range of values, around the design dimension, that are associated with normal manufacturing tolerances. Furthermore, the design dimensions may also vary from the illustrated dimensions, within the spirit of the invention. As illustrated in FIG. 4, base portion 336 has an outer diameter of about 25.527 mm. The length of speculum 300 is about 69.266 mm. Suction port 333 has an inner diameter of about 3 mm, and an outer diameter of about 4.038 mm. Plane A-A is shown in FIG. 4.

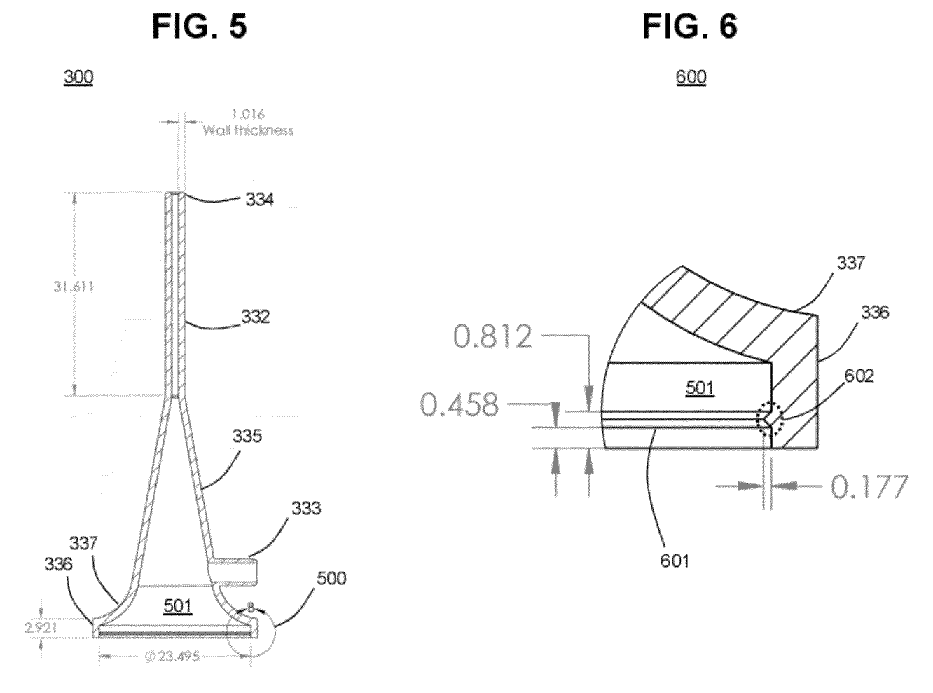

FIG. 5 illustrates a sectional view of speculum 300, with typical design dimensions, cut along plane A-A of FIG. 4. As illustrated, hollow cylindrical portion 332 has a length of about 31.611 mm and a wall thickness of about 1.016 mm. Base portion 336 may have an inner diameter of about 23.495 mm and an outer shoulder height of about 2.921 mm. A portion of base portion 336 is marked as detailed section 500.

FIG. 6 illustrates a detailed view 600 of section 500 marked in FIG. 5. Detailed view 600 illustrates a snap-on interface between otoscope head 501 and speculum 300, in particular with base portion 336 and second tapered section 337 of speculum 300. Otoscope head includes a circumferential groove 601 that mates with a circumferential notch 602 on an inner surface of base portion 336 when speculum 300 is mated to otoscope head 501. Exemplary design dimensions of circumferential groove 601 and matching circumferential notch 602 are as illustrated in FIG. 6.

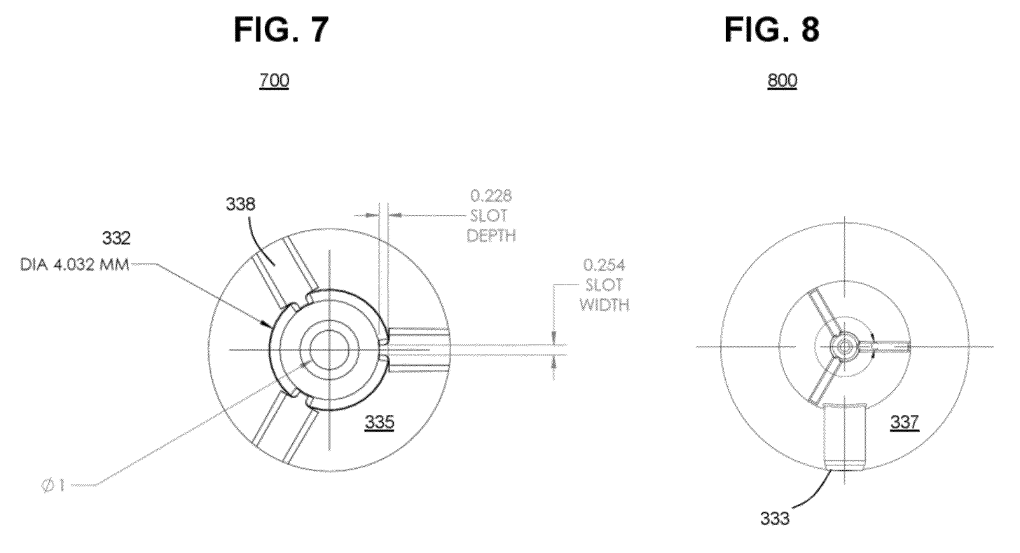

FIG. 7 illustrates a detailed top plan view of speculum 700 with typical design dimensions, in accordance with an embodiment of the present invention. In this embodiment, hollow cylindrical portion 332 has a nominal inner diameter of 1.0 mm and an outer diameter of about 4.032 mm. Grooves 338, in hollow cylindrical portion 332, have design dimensions of a width of about 0.254 mm and a depth of about 0.228 mm. Grooves 338 have a larger width in first tapered section 335.

FIG. 8 illustrates a top plan view of speculum 800, in accordance with an embodiment of the present invention. Speculum 800 of FIG. 8 is illustrated at less level of detail than speculum 700 of FIG. 7. Speculum 800 further illustrates second tapered section 337 and suction port 333.

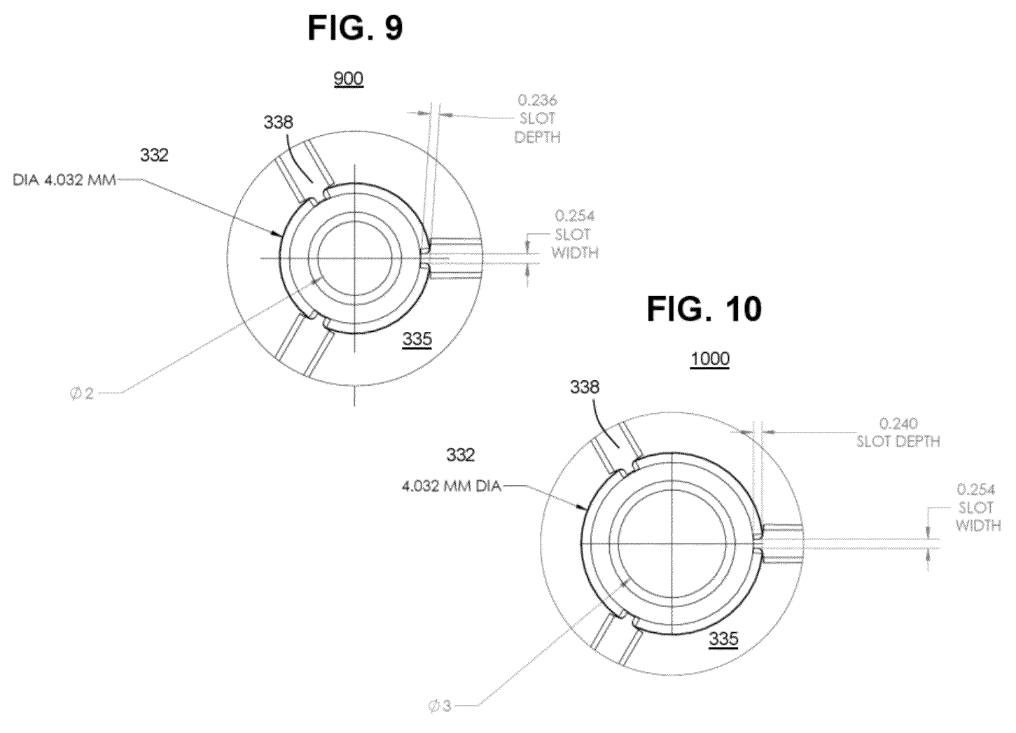

FIG. 9 illustrates a detailed top plan view of speculum 900 with typical design dimensions, in accordance with an embodiment of the present invention. In this embodiment, hollow cylindrical portion 332 has a nominal inner diameter of 2.0 mm and an outer diameter of about 4.032 mm. Grooves 338, in hollow cylindrical portion 332, have design dimensions of a width of about 0.254 mm and a depth of about 0.236 mm. Grooves 338 have a larger width in first tapered section 335.

FIG. 10 illustrates a detailed top plan view of speculum 1000 with typical design dimensions, in accordance with an embodiment of the present invention. In this embodiment, hollow cylindrical portion 332 has a nominal inner diameter of 3.0 mm and an outer diameter of about 4.032 mm. Grooves 338, in hollow cylindrical portion 332, have design dimensions of a width of about 0.254 mm and a depth of about 0.240 mm. Grooves 338 have a larger width in first tapered section 335.

While the foregoing is directed to embodiments of the present invention, other and further embodiments of the present invention may be devised without departing from the basic scope thereof. It is understood that various embodiments described herein may be utilized in combination with any other embodiment described, without departing from the scope contained herein. Further, the foregoing description is not intended to be exhaustive or to limit the present invention to the precise form disclosed. Modifications and variations are possible in light of the above teachings or may be acquired from practice of the present invention.

No element, act, or instruction used in the description of the present application should be construed as critical or essential to the invention unless explicitly described as such. Also, as used herein, the article “a” is intended to include one or more items. Where only one item is intended, the term “one” or similar language is used. Further, the terms “any of” followed by a listing of a plurality of items and/or a plurality of categories of items, as used herein, are intended to include “any of,” “any combination of,” “any multiple of,” and/or “any combination of multiples of” the items and/or the categories of items, individually or in conjunction with other items and/or other categories of items.

Moreover, the claims should not be read as limited to the described order or elements unless stated to that effect. In addition, use of the term “means” in any claim is intended to invoke 35 U.S.C. §112, ¶ 6, and any claim without the word “means” is not so intended.

What is claimed is:

1. A system to remove a foreign object from a body cavity, comprising:
a speculum, comprising:
a base configured to attach to an otoscope head;
a hollow tapered section, having a wide end coupled to the base, and a narrow end that is open; and
a hollow tube having a proximal end and an open distal end; the proximal end coupled to the narrow end of the hollow tapered section to form a hollow interior of the speculum;
a suction port coupled to a lateral side of the speculum, the suction port configured to provide access from outside the speculum to the hollow interior of the speculum;
a suction pump configured to provide a variably controlled suction strength; and
an interface to provide the variably controlled level of suction to the suction port.

2. The system of claim 1, further comprising a second tapered section, having a wide end coupled to the base, and a narrow end coupled to the wide end of the hollow tapered section.

3. The system of claim 2, wherein the second tapered section is detachably coupled to the wide end of the hollow tapered section.

4. The system of claim 1, wherein the variably controlled suction strength varies from about 0 mm Hg to about 1000 mm Hg.

5. The system of claim 1, wherein the hollow tube has a selectable inner diameter of about 1.0 mm to about 3.0 mm.

6. The system of claim 1, wherein the hollow tube has a selectable length of about 30 mm to about 50 mm.

7. The system of claim 1, wherein the hollow tube has a selectable inner diameter that is selected to cover from about 60% to about 90% of a size of the foreign object.

8. The system of claim 1, further comprising at least one transverse groove on an exterior surface of the hollow tapered section, coupled to a transverse groove on an exterior surface of the hollow tube.

9. The system of claim 1, wherein the suction pump further comprises: a user control of the suction strength; and a display of the suction strength.

10. The system of claim 1, wherein the body cavity is an ear canal.

11. A method to remove a foreign object from a body cavity, comprising the steps of:
selecting a hollow tube having an inner diameter less than a size of the foreign object, the hollow tube having a proximal end and a distal end opposite from the proximal end;
positioning the distal end of the hollow tube next to the foreign object;
providing a variably controlled suction strength through the proximal end of hollow tube, wherein the suction strength is sufficient to hold the foreign object to the distal end of the hollow tube; and
removing together the hollow tube and the foreign object at the distal end of the hollow tube, while maintaining the variably controlled suction strength.

12. The method of claim 11, further comprising the step of positioning the hollow tube by use of an otoscope coupled to the hollow tube.

13. The method of claim 12, wherein the hollow tube is coupled to the otoscope by use of a detachable base portion.

14. The method of claim 11, wherein the variably controlled suction strength varies from about 0 mm Hg to about 1000 mm Hg.

15. The method of claim 11, wherein the hollow tube has a selectable inner diameter of about 1.0 mm to about 3.0 mm.

16. The method of claim 11, wherein the hollow tube has a selectable length of about 30 mm to about 50 mm.

17. The method of claim 11, wherein the hollow tube has a selectable inner diameter that is selected to cover from about 60% to about 90% of a size of the foreign object.

18. The method of claim 11, further comprising the step of providing a flow of air to the distal end of the hollow tube by use of at least one transverse groove on an exterior surface of the hollow tube.

19. The method of claim 11, wherein the step of providing a variably controlled suction strength is by use of a suction pump comprising: a user control of the suction strength; and a display of the suction strength.

20. The method of claim 11, wherein the body cavity is an ear canal.
